(19) United States
(12) Patent Application Publication
(10) Pub. No.: US 2025/0258164 A1
(43) Pub. Date: Aug. 14, 2025

(54) DETECTION KIT FOR DETECTING BIOMOLECULES IN A REACTION TUBE BY MEANS OF INTEGRATION OF MOLECULAR BIOLOGICAL AMPLIFICATION OF NUCLEIC ACID AND DETECTION THEREOF BY MEANS OF LATERAL FLOW DETECTION

(71) Applicant: Fraunhofer-Gesellschaft zur Förderung der angewandten Forschung e. V., München (DE)

(72) Inventors: Natalia Sandetskaya, Leipzig (DE); Andreas Kölsch, Leipzig (DE); Kai Mattern, Leipzig (DE); Dirk Kuhlmeier, Leipzig (DE)

(21) Appl. No.: 18/960,049

(22) Filed: Nov. 26, 2024

(30) Foreign Application Priority Data
Feb. 12, 2024 (DE) 10 2024 103 902.7

(51) Int. Cl. G01N 33/53; C12Q 1/6844; G01N 33/543

(52) U.S. Cl. CPC G01N 33/5308; C12Q 1/6846; G01N 33/54388

(57) ABSTRACT

A point-of-care detection kit for nucleic acids, comprising a reaction tube (1), at least one miniaturized lateral flow strip (2), a holder for the miniaturized lateral flow strip (3), and amplification reagents for loop-mediated isothermal amplification (4), wherein the at least one miniaturized lateral flow strip (2), the holder (3) and the amplification reagents (4) are integrated into the reaction tube.

### TECHNICAL FIELD

The present invention is in the technical field of detection systems for nucleic acids.

### BACKGROUND AND PRIOR ART

Modern point-of-care testing requires test systems which are efficient, precise, easy to handle and reliable and which provide all the necessary information and avoid interfering factors such as contamination. This requires integration of highly sensitive molecular diagnostic detection methods into suitable test systems. Examples of the areas of application for these test systems include human and veterinary medicine, food analysis, various areas of hygiene, and forensic applications. The detection of the desired biomolecules generally requires complex molecular biological methods for amplification of the nucleic acid in a contamination-free environment with the aid of complex devices and also microfluidic methods and devices for detection of the nucleic acids. However, for point-of-care applications, they are not available on-site and are often far too expensive.

Detection by means of lateral flow tests can allow device-free detection of analytes; however, this format is based on manipulating liquid samples and test strips (addition of a special buffer and application of the sample to test strips or test cassettes). However, this open handling of the sample cannot be used in the case of a molecular biological test: Especially contact between the sample and the environment following nucleic acid amplification is associated with a high risk of contamination for other tests carried out in parallel, i.e., the uncontrolled transfer of amplification products to contact surfaces can occur, thereby leading to contamination in other tests. The minimal transfer of amplification products to other tests can generate false-positive results, thus invalidating these tests without this being registered by the user. A molecular diagnostic test for point-of-care applications with lateral flow detection can thus only be used reliably if the detection takes place in a completely closed system without reaction products being further manipulated.

Only two systems allowing lateral flow-based detection of DNA products are known to date:

1) Test cartridge from SelfDiagnostics Deutschland GmbH (MULTITEST COVID-19—Selfdiagnostics) (FIG. 4A), which integrates integrated isothermal amplification of nucleic acids and detection thereof on a lateral flow base. The two processes proceed in a closed microfluidic chip. The detection buffer is pre-stored in a blister on the cartridge; addition of said buffer is realized by pressing on the blister. However, the cartridge is quite large (the size of a hand) and also contains batteries for the heating elements (the amplification proceeds at ˜65° C.). Regular use of said cartridge in point-of-care products is not environmentally sustainable. The costs of producing said cartridge are also very high owing to the complexity of the system.

2) Detection cartridge from UStar (Nucleic Acid Probes (fragments) Detection Device_USTAR BIOTECHNOLOGIES (HANGZHOU) LTD. (bioustar.com) (FIG. 4B)). This system does not contain integrated nucleic acid amplification, but it does allow closed detection of externally amplified DNA products: The UStar cartridge contains a reservoir with detection buffer and contains a mechanism for mixing the products from a reaction tube with the buffer and for transferring the mixture onto the integrated test strip. This system is also the size of a hand, requires substantial quantities of plastics for manufacture thereof, and costs ˜€13 per detection.

The pre-existing detection systems cannot be used for DNA amplification without access to laboratory equipment, they require environmentally unfriendly components, they are expensive and/or their execution requires manual steps to transfer nucleic acid-containing liquids that encourage contamination outside the controlled laboratory environment. The described detection systems are not suitable for areas of application with a very large number of tests/high sample throughput (e.g. mass testing, food analysis).

It is an object of the present invention to develop a sensitive point-of-care test system for the amplification and subsequent detection of nucleic acids, wherein the entire reaction is carried out in a closed standard reaction tube in order to avoid contamination and to reduce sample amounts, liquid volumes and work steps and the detection of nucleic acids does not require any laboratory equipment.

According to the independent claims, this object is achieved by a detection system for nucleic acids that is completely integrated into a standard reaction tube (FIGS. 1A and 1B). Said system consists of a standard reaction tube (1) (e.g. 0.5 ml tube) which contains a miniaturized lateral flow strip (mini-LFT) (2) and the holder for the miniaturized lateral flow strip (mini-LFT) (3) and which has been filled with amplification reagents, preferably in dried form, for loop-mediated isothermal amplification (LAMP) (4). FIG. 1B shows a conceptual design for integration into a conventional laboratory tube. The insert is produced together with the cap and connected thereto in a two-component injection moulding process. This simplifies the handling of the system and reduces production costs.

### BRIEF DESCRIPTION OF THE DRAWINGS

FIG. 1 shows in A: a schematic illustration of the reaction tube, and in B: a conceptual design of the insert cap and its integration into a reaction tube.

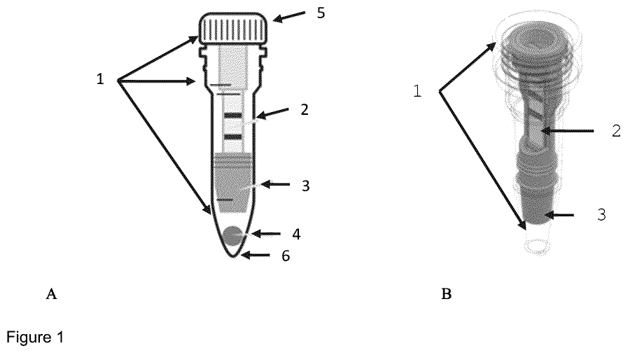

FIG. 2 shows a schematic illustration of the use of the point-of-care test system.

FIG. 3 shows a photo of a positive test result (visible strip for test (T) and for control (C)) versus a negative test result (visible strip for control (C) only).

FIG. 4 shows the relative sizes (compared to the size of a hand) of the described integrated systems (A: SelfDiagnostics, B: UStar) vs CampyTube (C). Sources:

A: https://www.linkedin.com/posts/selfdiagnostics_covid-testing-activity-6963402026651201536-fDzg?utm_source=share&utm_medium=member_desktop,

B: https://www.twistdx.co.uk/product/u-star-disposable-nucleic-acid-lateral-flow-detection-units-pack-of-20/,

C: detection kit according to the invention.

### DESCRIPTION OF THE INVENTION

The subject matter of the present application is apparent from the independent claims, and advantageous optional configurations are stated in the dependent claims.

The mini-LFT is a miniaturized version of a classic lateral flow test strip. DNA products are detected as a result of binding thereof to functionalized regions of the membrane. Detection of said DNA products requires that they be labelled with special labels (the labelling with labels is carried out during amplification through the labelling of appropriate primers and is prior art).

The holder for the miniaturized lateral flow strip (mini-LFT) ensures optimal spatial separation of the reaction and the lateral flow strip, thereby preventing premature activation of the lateral flow strip. Furthermore, the holder prevents the liquid from evaporating during the reaction, and optimal transfer of the liquid onto the test strip is ensured.

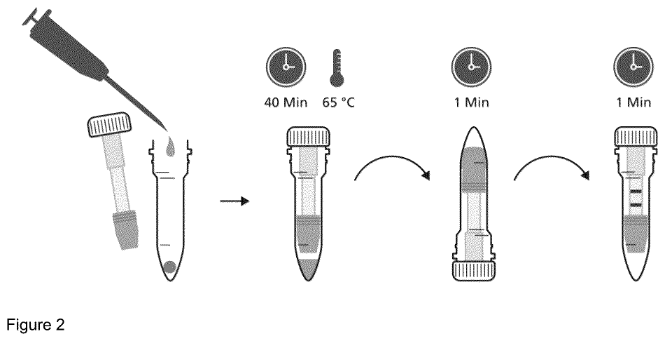

The test method (FIG. 2) comprises adding the sample to the reaction tube, followed by tightly closing the tube using the insert cap which carries the mini-LFT, incubating the sample with suitable reagents at the defined temperature for DNA amplification, and turning the reaction tube over in order to activate the mini-LFT (fluidic transport). The results are read with the naked eye, since the test and control bands are visible through the transparent wall of the reaction tube.

All the test components, from the preferably dry amplification reagents to the mini-LFT, are contained in a single tube which is closed after the liquid sample has been added and which need never be reopened. This completely prevents carryover of contamination, and the system is very safe and simple to use at the point of care. The system can be integrated into different reaction tubes of different volumes with minimal effort.

The detection method according to the invention is stated in independent claim 6, and steps a) to d) are preferably carried out in the order according to the claim.

The integrated lateral flow system for molecular point-of-care testing has been developed for an example application, the detection of Campylobacter bacteria in meat production plants, and is therefore named CampyTube. However, the system is very versatile and can be adapted for various applications.

The integrated test system-also referred to here as detection kit-CampyTube may be preferably fitted into a standard 0.5 ml tube having a screw cap. The detection is based on isothermal amplification of a pathogen-specific DNA sequence as part of loop-mediated isothermal amplification (LAMP). The sample may be, for example, collected as a surface swab for hygiene control and eluted in a LAMP-compatible lysis buffer. Other sample types, for example liquids such as rinsed meat samples, are also premixed with lysis buffer.

Following lysis, the sample (25 μl) is added to the reaction tube containing dry reagents for LAMP. This step may be carried out with a drip tube. The LAMP primers are labelled in order to allow lateral flow product detection. In our system, one of the forward primers bears biotin, whereas one of the reverse primers is FAM-labelled. The resultant DNA amplicons thus contain both labels. The lateral flow detection is based on the gold nanoshells conjugated with anti-FAM mouse antibodies reacting with the label on the LAMP product, while the biotinylated end of the product is captured on a streptavidin test line on the lateral flow membrane. The excess of anti-FAM nanoshells is captured on a control line (anti-mouse goat antibody).

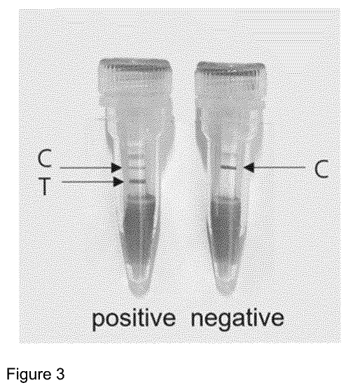

For the detection of Campylobacter, the analytical sensitivity is currently ˜60 copies per reaction. Campylobacter was detected in rinsed meat samples and surface swabs by means of the test system. 17 samples of the 60 samples tested contained >1000 CFU/g meat according to a microbiological reference method (this is the quality control threshold according to DIN EN ISO 10272-2). These 17 positive samples were identified as positive by the inventive integrated test system (CampyTube) and established qPCR (comparative system). Fifteen additional meat samples contained lower pathogen loads (between 10 and 1000 CFU/g, 66.7%). 10 out of 15 of these positive samples were detected by the integrated test system CampyTube. With the aid of qPCR (comparative system), 100% (15 out of 15) of the positive samples were detected. In the case of 28 samples which contained <10 CFU/g meat or were negative in microbiological tests, 5 (17.9%) samples were positive with CampyTube and 14 (50%) were positive with qPCR. The current sensitivity of the integrated test system CampyTube allows the use of the test system in a practically relevant range. Examples of positive and negative test results can be found in FIG. 3.

The integrated test system is the first system that seamlessly combines molecular nucleic acid amplification and lateral flow detection in a standard laboratory tube in a fully integrated one-step test system. This system is environmentally sustainable owing to the reduced amount of plastic, since the amount of plastic in the test system is only 15% of the amount used in typical LFT cassettes (˜0.95 g versus ˜6.35 g).

The test system is preferably closed with the cap, also referred to here as closure, which accommodates the integrated mini-LFT and its holder. The mini-LFT is fixedly integrated in a special insert (part of the cap). The insert may also be used separately from the cap. The inserts may be produced, for example, in silicone casting with the aid of 3D-printed moulds. In the present concept, the cap and the insert are provided as an industrially manufactured piece. A one-piece format makes the use of the test safer, more reproducible and simpler.

The special insert ensures the optimum depth of positioning of the mini-LFT in the reaction tube directly above the liquid LAMP reaction mixture and prevents premature contact thereof with the mini-LFT. Neither sudden dropping of the reaction tube nor moisture condensation during the heating step (LAMP scheme: 40 min at 65° C.) can lead to premature activation of the mini-LFT, since there is a special funnel-shaped spacer in the bottom of the insert. Once the LAMP amplification has ended, the mini-LFT can be activated by turning the tube over and gently flicking the wall of the tube. This breaks the surface tension of the liquid, and the liquid containing the amplified specific DNA sequence flows towards the mini-LFT.

A preferred embodiment of the mini-LFT has dimensions of 25 mm×3 mm. Miniaturization allows omission of the sample pad to save space without compromising the analytical efficiency of the test. The mini-LFT consists of a conjugate pad, a nitrocellulose membrane containing the test and control lines, and a wick pad. Owing to the miniaturized dimensions of the mini-LFT and the optimized gold nanoshell conjugate, the detection time is very short. The test and control bands are visible with the naked eye within about a minute. In comparison, conventional LFTs require up to 10 minutes for visualization. The volume of 25 μl is sufficient for the LAMP reaction and effective detection in the mini-LFT. This is a distinct advantage of the test system: There is no need to add buffer, and so the reaction tube remains closed following the primary addition of the sample. This completely eliminates the risk of carryover contamination. For these reasons, the test system is perfectly suited to point-of-care use by non-professional users.

The direct integration of the mini-LFT into a standard 0.5 ml disposable laboratory tube and the material-saving (˜3× less nitrocellulose) miniaturized LFT make the system very inexpensive and compatible with all standard heating blocks. This format is particularly advantageous for areas of application with high throughput or low resources.

The advantages of the test system according to the invention are:

1) Completely closed format with seamless integration of a molecular assay and detection.

2) Small reaction volumes (e.g. 25 μl) are sufficient for detection: There is no need to add buffer (unlike conventional test strips, where another 100-200 μl of detection buffer must be added to the sample).

3) Device-free detection.

4) Compatibility with standard heating blocks for amplification at a specific temperature: No need for a complex device and batteries.

5) Cost-effective: The specially produced plastic part comprises only the insert cap, which can be produced in relatively large quantities as an element of simple construction (compared to complex large cartridges comprising a multitude of individual parts to be produced).

6) Material-saving: ˜3× less nitrocellulose to produce the mini-LFT; up to 80% less plastic compared to conventional LFT test cassettes.

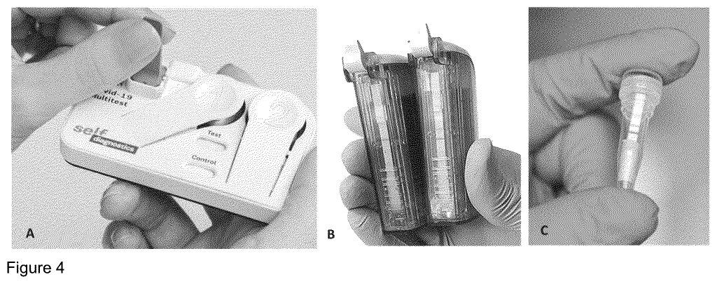

7) Small overall dimensions and light weight compared to the other systems presented (FIG. 4). This makes logistics much more cost-efficient.

Therefore, a detection kit for nucleic acids, preferably DNA, a method for detecting biomolecules, preferably nucleic acids, particularly preferably DNA, and the use of the detection kit for detection of biomolecules, preferably nucleic acids, particularly preferably DNA, form part of the present invention.

1. A point-of-care detection kit for nucleic acids, comprising a reaction tube, at least one miniaturized lateral flow strip, a holder for the miniaturized lateral flow strip, and amplification reagents for loop-mediated isothermal amplification, wherein the at least one miniaturized lateral flow strip, the holder and the amplification reagents are integrated into the reaction tube.

2. The detection kit according to claim 1, further comprising a closure designed for reversible closure of the reaction tube, the miniaturized lateral flow strip and the holder being attached to the closure such that the miniaturized lateral flow strip and the holder extend away from the closure in the direction of a bottom of the reaction tube and the miniaturized lateral flow strip is directly attached to the closure and the holder is directly attached to the miniaturized lateral flow strip.

3. The detection kit according to claim 1, wherein the reaction tube has a shape which is at least partly tapered in the direction of the bottom and the amplification reagents are present at the bottom of the reaction tube.

4. The detection kit according to claim 1, wherein the amplification reagents for loop-mediated isothermal amplification are present in the reaction tube in dried form.

5. The detection kit according to claim 1, wherein the holder is designed such that, when the reaction tube is closed, the holder separates the reaction tube into at least two reaction compartments, such that the amplification reagents are present in a bottom-side reaction compartment and the miniaturized lateral flow strip is arranged in a closure-side reaction compartment.

6. A method for detecting nucleic acids, comprising:
a) providing a detection kit according to claim 1,
b) adding the sample to be analysed to the reaction tube to the amplification reagents,
c) carrying out incubation at a specified temperature and for a specified time, and
d) evaluating the results.

7. The method according to claim 6, further comprising:
e) directly after step a), removing the closure,
f) directly after step b), closing the reaction tube by means of the closure,
g) directly after step c), moving the reaction tube to a substantially vertical position, such that the bottom of the reaction tube is pointing substantially upwards, and
h) directly after step g), moving the reaction tube to a substantially vertical position, such that the bottom of the reaction tube is pointing substantially downwards.

8. The method according to claim 7, wherein an amplification and the evaluation of the results take place in the reaction tube and, after step f), material, in particular sample liquids and/or reagents, are not added to and/or removed from the reaction tube.

9. (canceled)

10. A method of detecting DNA in a closed reaction environment comprising using the detection kit of claim 1.
